(19) United States
(12) Patent Application Publication
(10) Pub. No.: US 2019/0252045 A1
(43) Pub. Date: Aug. 15, 2019

(54) SYSTEM AND METHOD OF ACQUISITION, TRANSMISSION AND PROCESSING DATA RELATED TO BIOLOGICAL FLUIDS

(71) Applicant: Antonio Valentini, Roma (IT)

(72) Inventor: Marco Flavio Michele VISMARA, Roma (IT)

(21) Appl. No.: 16/342,037

(22) PCT Filed: Oct. 17, 2017
(86) PCT No.: PCT/IB2017/056424

§ 371 Date: Apr. 15, 2019

(30) Foreign Application Priority Data
Oct. 17, 2016 (IT) 102016000103909

(51) Int. Cl. G16B 45/00; A61B 5/1455; A61B 5/00; G06N 20/00

(52) U.S. Cl. CPC G16B 45/00; G06N 20/00; A61B 5/0075; A61B 5/1455

(57) ABSTRACT

A system for acquisition, transmission and processing data related to biological fluids comprising at least one support configured to acquire at least one biological sample of a patient, at least one infrared spectrometer configured to acquire the infrared spectrum of the sample, at least one server connected to the infrared spectrometer by internet and configured to receive the spectrum obtained by the infrared spectrometer, performing a step of post-processing and analysis of received data.

The system comprises:
at least one device comprising a camera and an OCR system configured to acquire an image of the support and to decode personal data contained in it and send them to the server, to receive data analyzed from the server and to display these data; and
a private block/hybrid Cloud containing the server, a login system, a management program for the historical analysis carried out, a system for sending a draft report to an accredited Physician and/or Biologist and/or Chemist and/or Veterinarian.

### CROSS-REFERENCE TO RELATED APPLICATIONS

This application is a U.S. National Phase application under 35 U.S.C. 371 of International Application No. PCT/162017/056424, filed on Oct. 17, 2017, and published in English as WO 2018/073730 A2 on Apr. 26, 2018, which claims priority to Italian Patent Application No. 102016000103909, filed on Oct. 17, 2016. The entire disclosures of the above applications are incorporated herein by reference.

### FIELD

The present invention relates to a system of acquisition, transmission and processing data related to biological fluids.

The present invention is also relating to a method of acquisition, transmission and processing data related to biological fluids.

In particular, the present invention relates to a system and to a method of acquisition, transmission and processing data related to biological fluids, such as blood chemistry data for remote clinical analysis.

### BACKGROUND

This section provides background information related to the present disclosure which is not necessarily prior art.

As it is well known, clinical chemistry tests allow to detect and quantify the substances present in body fluids, as blood, urine, tears, cerebrospinal fluid, milk, sperm, sputum, and others. These analyses are useful for diagnostic purposes and in discovering anomalies and are carried out using various chemical analysis techniques to determine the amount of various organic and inorganic substances circulating in the body of human and veterinary patients.

The blood chemistry analyses are currently carried out in laboratories. The medical or paramedical staff performs a patient's venous blood sampling (or other biological sample), usually in the morning to achieve better standardization and reduce biological variability. The collected blood is stored and sent to the analytical laboratory. The laboratory performs the required analyses and issues a report that must then be transmitted and transported to the patient and his/her/its physician.

Hence, the acquisition, transmission and processing of blood chemistry data occur by means of systems and methods that involve high costs and timings, as well as the necessity of the direct presence of medical and/or paramedic personnel.

A known chemical analysis technique is Infrared Spectroscopy (IRS) that identifies the substances present in a sample. Such an analysis technique has the advantage of being particularly economical and of not requiring the use of chemical reagents.

However, the application of IRS to clinical analysis has the limit of not being able to analyze liquid samples. In addition, the IRS itself presents little accuracy of the results obtained.

### SUMMARY

This section provides a general summary of the disclosure, and is not a comprehensive disclosure of its full scope or all of its features.

The purpose of the present invention is to provide a system and a method of acquisition, transmission and processing data related to biological fluids, such as blood, that can be cheap, simple and precise, therefore having characteristics such as to overcome the limitations which still affect the current systems and methods for acquisition, transmission and processing related to biological fluids with reference to the prior art.

A further aspect of the present invention is to provide a system and a method of acquisition, transmission and processing data related to biological fluids which allows to perform the taking of a biological sample, as blood, without the assistance of medical, veterinary or paramedical personnel and processing of the data obtained at a distance by IRS.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

For a better understanding of the present invention, a preferred embodiment is now described, purely by way of non-limiting example, with reference to the accompanying drawings in which:

FIG. 1 shows a schematic diagram of a system of acquisition, transmission and processing data related to biological fluids, according to the invention;

FIG. 2 shows a diagram of a portion of the system of acquisition, transmission and processing data related to biological fluids, according to the invention;

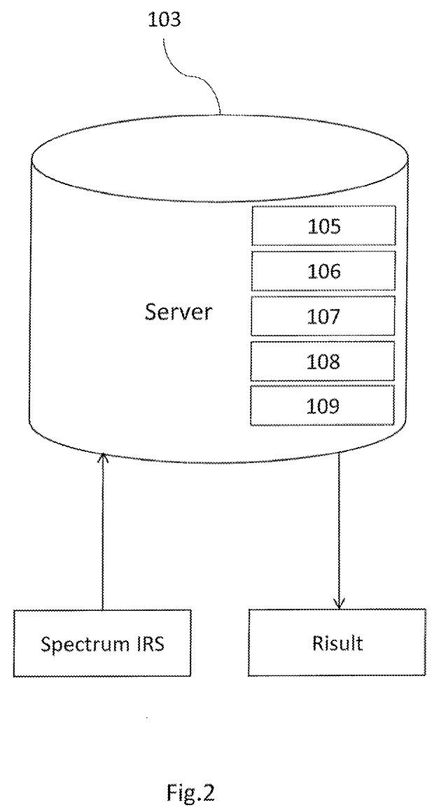

FIG. 3 shows a schematic diagram of the method of acquisition, transmission and processing data related to biological fluids, according to the invention.

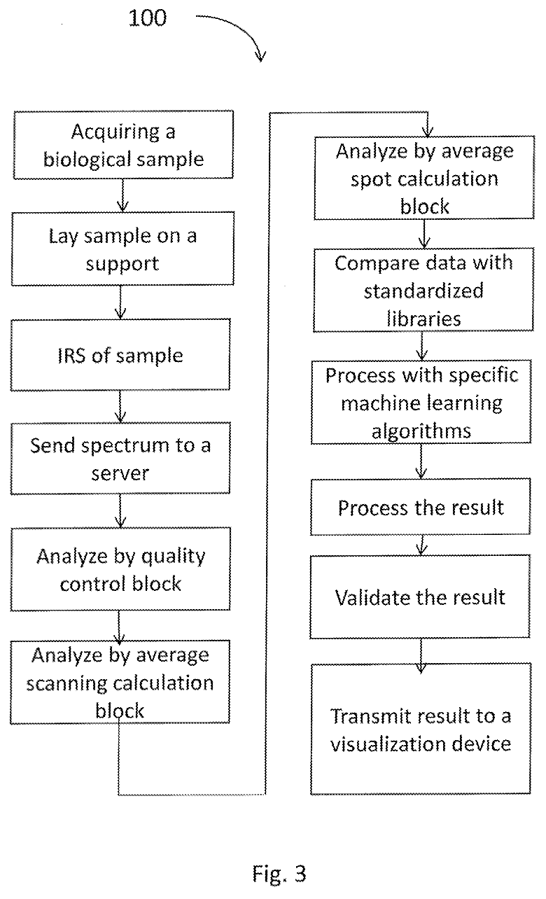

### DETAILED DESCRIPTION

Example embodiments will now be described more fully with reference to the accompanying drawings.

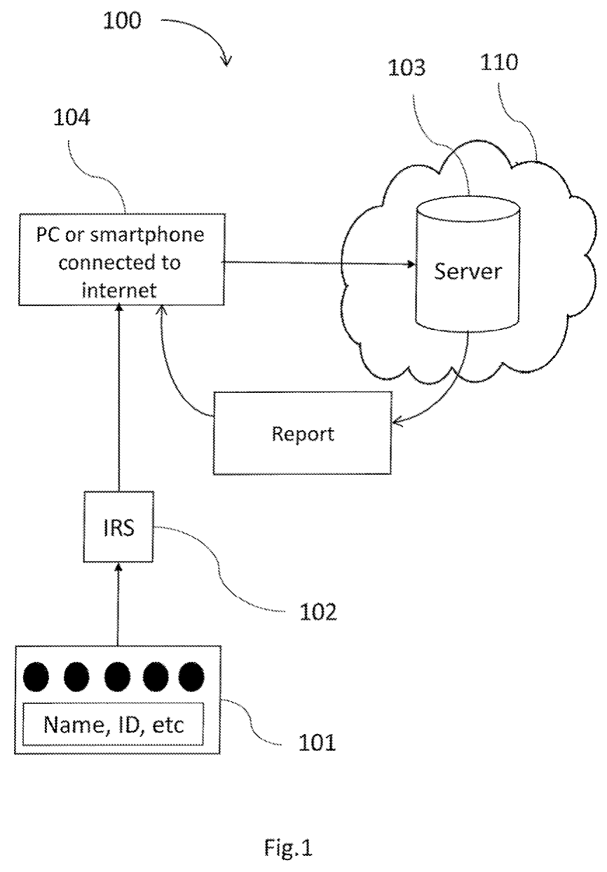

With reference to these figures, and in particular to FIG. 1, a system 100 of acquisition, transmission and processing data related to biological fluids is shown, according to the invention.

More in detail, the system 100 of acquisition, transmission and processing data related to biological fluids comprises a support 101, an infrared spectrometer 102, a server 103, or a servers cluster, to which is connected the infrared spectrometer 102 and a device 104 configured for the transmission, reception and viewing of the analyzed data of the acquired infrared spectrum and also able to take pictures, needed for acquired image recognition/OCR data by server 103.

According to an aspect of the invention, as shown in FIG. 1, the infrared spectrometer 102 is connected to the device 104 so that the spectrum resulting from the analysis of the sample considered can be sent via internet connection to said server 103, or cluster of servers, according to the needs of analysis.

According to an aspect of the invention, the infrared spectrometer 102 is connected directly to server 103 by means of an Internet connection in such a way as to send to the server 103 the spectrum resulting from the analysis of the sample considered. According to an aspect of the invention, the server may be a physical or a virtual machine.

According to an aspect of the invention, the device 104 comprises a camera and an image recognition/OCR system for capturing a picture related to the support 101 and decoding the master data and possibly the signature of the patient therein comprised. These data are sent to the server and are uniquely associated with the analyzed blood sample.

According to an aspect of the invention, said image recognition/OCR system automatically verifies the captured picture of the support 101, to detect and compare regularity, color and dimensional spot's data. In the eventuality in which the OCR would detect an inconsistency or an error in the captured picture, the system will request an additional picture of the support 101 and subsequently, in case of further inconsistency or error, to exclude the first sample and to provide a newly collected one.

According to an aspect of the invention, the processing server 103 is contained in a private/hybrid cloud 110 block. This block 110, in addition to server 103, contains a logging system, a management for the analysis history, a system to submit the draft report to the physician/chemist/biologist accredited for signing electronic signature.

Advantageously, the cloud system allows to acquire additional computational power dynamically on demand, in order to render the system resilient to any peaks of data processing request.

According to an aspect of the invention, the support 101 is made up of an absorbent paper, preferably of the commercially available type as Whatman 903 protein saver, or equivalent.

According to an aspect of the invention, the support 101 is constituted by a card on which the patient identification data can be specified by handwriting and it is associated to a barcode. In order to comply with eventual regulations regarding privacy or informed consent, patients can sign this card to express agreement.

The support 101 is capable of acquiring one or more biological samples of the patient. The support 101 is also capable of being scanned by the IRS by the use of the infrared spectrometer 102 so as to allow detection of the chemicals contained in the considered spot.

Advantageously according to the invention, the support 101 allows to analyze a blood sample by means of IRS spectroscopy.

The infrared spectrometer 102 acquires the blood sample and analyzes it by performing a set number of scans for each patient's blood spot. For example, the scans performed are 32 for 3 spots per patient.

The spectrum data obtained by the scans is automatically sent to the server 103. The server 103 is configured to perform the post-processing phases and the analysis of the received data, and send them in real time, after validation, to the device 104 located at the patient and/or his doctor.

Advantageously according to the invention, the results of the clinical chemistry analysis performed on the sample in an economical and simple manner are transmitted in real time to the doctor and/or the patient.

Advantageously according to the invention, clinical chemistry data are verified and controlled to increase the reliability of the IRS analysis performed.

Advantageously according to the invention, the system and method can be applied on humans or animals.

According to an aspect of the invention, the server 103 comprises a quality control block 105, a scans' average calculation block 106, a spots' average calculation block 107 of the patient, one or more standardized library 108, a block 109 including specific machine learning algorithms.

According to an aspect of the invention, the support 101 comprises a bar code and a pre-perforated section so as to separate a first part containing the blood samples and a second part containing the patient's data.

Advantageously, separating the support 101 in two parts along the pre-perforated section, the barcode/qrcode is divided into two symmetrical halves, so it is still readable on both sides. The bar code and a serial number associated with the support 101 are uniquely associated with the patient by capturing the image by means of the device 104.

Advantageously according to the invention, a first part of the support 101 containing the blood spots may also be sent by mail or courier to a laboratory for analysis to be subjected to types of analysis that cannot be performed by IRS, such as genetic or infectious tests, while a second part of the support 101 containing the barcode, or qrcode, and the master data would constitute a receipt and traceability document of the sent sample.

Advantageously, the first part of the support 101 containing the blood spots is thus easily transportable and invisible because it is thin and light and also anonymous.

As previously said, the present invention also relates to a method of acquisition, transmission and processing data related to biological fluids.

In particular, the method of acquisition, transmission and processing data related to biological fluids data comprises the steps of:

acquiring at least a patient's biological sample; in case of a blood sample, for example, puncturing a finger;

placing the blood sample on a support 101;

using device 104 image recognition/OCR system for capturing a picture related to the support 101;

sending via Internet connection the obtained data to a private hybrid cloud 110 comprising a server 103;

decoding the master data and possibly the signature of the patient therein comprised.

submitting back the authorization to proceed with IRS spectroscopy;

submitting the support 101 to IRS spectroscopy by means of an Infrared Spectrometer 102;

obtaining the IRS spectrum acquisition of the sample;

sending via Internet connection the obtained spectrum to a private hybrid cloud 110 comprising a server 103;

analyzing data using a quality control block 105;

analyzing the data using a calculation block of the average of scans 106;

analyzing data using a block 107 of computing average of the spots of each patient;

comparing data with one or more standardized libraries 108;

processing the results of the chemical analysis on the analyzed sample by means of a block 109;

validating the result by means of a physician or an accredited biologist or chemist;

transmitting in real time the result of the analysis of blood chemistry to a display device 104 and/or in paper or electronic form.

According to one aspect of the invention, the step of depositing the biological sample on support 101 may be repeated several times as the patient repeats the operation for one or more spots.

According to another aspect of the invention, the server 103 is the “machine learning” unit of the system. Here complex data analysis can be done to solve pattern recognition problems as handwriting recognition, quality control of sample loading, and IRS spectra analysis.

According to another aspect of the invention, the steps of sending the spectrum obtained to the server 103 and of transmitting in real time the result of the clinical chemistry analysis to the device 104 occur via an internet connection.

According to an aspect of the invention, the step of processing the result of the blood chemistry analysis on the examined sample takes place by means of the application of algorithms of “machine learning” optimized for the analyte examined, not excluding the application of more ad hoc methods for a more accurate prediction. To this scope machine learning methods (“machine learning”) will be used alone or in combination, depending on the outcome of fractures “selection” implemented ad hoc.

By way of example, the analyte TSH can be analyzed with Support Vector Machines algorithms. The analyte T4 may instead be analyzed with Random Forest techniques.

Advantageously, the system and the method of acquisition, transmission and processing of data related to biological fluids according to the invention are cheap and accurate.

Advantageously, the method of acquisition, transmission and processing data related to biological fluids according to the invention, by means of the phases of analysis and processing carried out by the server 103, allows to solve the problem of the poor accuracy of the IRS technique and can make advanced calculations, access to database reference and apply specific mathematical models, based on machine learning and artificial intelligence, to the obtained data.

Advantageously according to the invention, the server 103 utilizes an algorithm optimized for each analyte in the analysis of clinical chemistry data.

Advantageously, the system and method of acquisition, transmission and processing data related to biological fluids according to the invention, are versatile, presenting a wide range of applications.

Among the applications of the system and method according to the invention are, for example, neonatal screening; clinical analysis at “points of care” such as pharmacies, clinics analysis at medical clinics; home clinical analysis, self-testing, personal testing; field testing, as on site testing in emergency, police controls, military environment; telemedicine applications for isolated communities, as intercontinental ships personnel or remote areas inhabitants.

Advantageously, the system and method of acquisition, transmission and processing of data related to biological fluids according to the invention, can be performed without using of chemical reagents and no toxic waste is generated from the analytical process.

Advantageously, the system and method of acquisition, transmission and processing of data related to biological fluids according to the invention, can be performed without analysis done by medical or paramedical personnel, without sending the sample to a laboratory for analysis and without waiting the processing of the analysis.

Advantageously according to the invention, the system and the method of acquisition, transmission and processing data related to biological fluids are low cost.

Advantageously, the system and method of acquisition, transmission and processing data related to biological fluids according to the invention are of low environmental impact, not involving the use of chemical reagents.

Finally, it is clear that the system and the method of acquisition, transmission and processing data related to biological fluids described and illustrated herein can be subject to modifications and variations without thereby departing from the scope of the present invention, as defined in the appended claims.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are to be regarded as a departure from the disclosure, and all such modifications are intended to be included within the scope of the disclosure.

1. A system for acquisition, transmission and processing data related to biological fluids comprising at least one support configured to acquire at least one biological sample of a patient, at least one infrared spectrometer configured to acquire the infrared spectrum of the biological sample, at least one server connected to the infrared spectrometer by internet and configured to receive the spectrum obtained by the infrared spectrometer, performing a step of post-processing and analysis of received data, characterized in comprising:
at least one device comprising a camera, and an OCR system configured to acquire an image of the support and to decode personal data contained in the support and send them to the server to be uniquely associated to the analyzed sample, the device being also configured to receive data analyzed from the server and to display these data; and
a private block/hybrid cloud containing the server, a login system, a management program for the historical analysis carried out, a system for sending a draft report to be validated to an accredited Physician and/or Biologist and/or Chemist and/or Veterinarian, before sending it back to the device.

2. The system for acquisition, transmission and processing data related to biological fluids according to claim 1, wherein the support is a Guthrie paper on which the patient identification data are specified.

3. The system for acquisition, transmission and processing data related to biological fluids according to claim 1, wherein the server comprises at least one quality control block, at least one scans' average calculation block, at least one spots' average calculation block, at least a standardized library, and at least one block comprising machine learning algorithms.

4. A method for acquisition, transmission and processing data related to biological fluids comprising the steps of:
acquiring at least a patient's biological sample;
acquiring the infrared spectrum of the biological sample;
making the acquisition of the IRS spectra of the sample by means of at least one server; and
sending the spectrum obtained to a device configured to transmit the infrared spectrum acquired in the at least one server, receive data analyzed from the server and display said data, characterized in comprising the steps of:
checking the quality of the analyzed data;
calculating scans' average;
calculating spots' average of a single patient;
comparing the data with one or more standardized libraries;
processing the results of the clinical chemistry analysis on the examined sample by machine learning algorithms;
validating the results by a Physician and/or Biologist and/or Chemist and/or Veterinarian; and
transmitting results of the clinical chemistry analysis in real-time to a display device.
